(19) United States
(12) Patent Application Publication
(10) Pub. No.: US 2006/0161116 A1
(43) Pub. Date: Jul. 20, 2006

(54) Protective cover for arterial needle

(76) Inventors: Allan F. Willis, Marlton, NJ (US); Sedo Cha, Medford, NJ (US)

Correspondence Address: Hollstein Keating Cattell Johnson & Goldstein P.C.;Willow Ridge Executive Office Park, Suite 301, 750 Route 73 South, Marlton, NJ, 08053, US

(21) Appl. No.: 11/039,333

(22) Filed: Jan. 19, 2005

(51) Int. Cl. A61M 5/00

(52) U.S. Cl. 604/263

(57) ABSTRACT

A protective cover is provided which is effective in covering the needle tip, but which also can be retracted along the needle shaft to uncover and reopen the needle without interference with the cover or causing contact damage between the cover and the needle tip. The cover has a sheath section with a through channel into which the shaft is slideably received. The cover's sheath section travels substantially along the shaft. This movement is controlled by an actuator control knob connected to a biased locking spring clip or leaf spring located within the sheath section. The locking spring has dual openings through which the shaft travels. When the cover is secured in position on the needle shaft, e.g. when the needle is to be used or when the needle tip is enclosed, the openings are slightly misaligned. This causes locking of the resilient shaft within the protective cover. Pressing the control knob aligns the openings, which unlocks the needle tip and allows the cover to be slid along the shaft.

### BACKGROUND OF THE INVENTION

The need to ensure that medical needles, including arterial needles, are properly sheathed when not in use is of critical importance in the healthcare industry. It is necessary that provision is made to safely handle needles both as they are being used and afterward, during the disposal process. Of course, it is the extremely sharp tip of the needle which creates a puncture hazard; but if the needle has been contaminated and the skin is pricked or even touched, there is also a real risk of exposure to disease, especially when dealing with highly contagious diseases such as AIDS and hepatitis. This risk of contamination is especially present during the disposal of needles.

To address this hazardous situation, the medical appliance industry has developed protective covers or guards which are designed to be positioned over the sharp tips of needle shafts. Immediately after a needle is used, a protective cover is placed over the tip, before the health care professional handles the needle any further. When being disposed, the needle remains enclosed. The hazards presented by sharp, contaminated needles are drastically reduced.

There are many different protective covers or guards in the industry. See, for example, those disclosed in U.S. Pat. Nos. 4,929,241, 5,098,401, 5,344,408, 6,001,080, 6,210,373, and 6,623,458. However, although different, the various prior art protective devices have one thing in common; they are generally all designed for single usage applications. In other words, needles are to be used one time and once the covers discussed in the prior art are positioned over the needle tips, the covers are not normally intended to be removable so that the needles can be re-used. In most cases, the needles are then designed to be discarded after a single use.

This presents a glaring shortcoming, because often times it may be necessary or advantageous to withdraw and then reuse the needles. Nonetheless, safety dictates that needle tips be covered when not in use. Yet once a cover encloses a tip, it cannot or is not designed to be removed and the needle must be discarded—compelling the use of another needle. The protective cover for an arterial needle discussed in co-pending application Ser. No. 10/942,705, addresses this situation by providing a cover which effectively encloses the tip of the needle and which also can be retracted along the needle's shaft to uncover the tip of the needle. The subject invention takes this improvement a step further by preventing possible damage to the tip of the needle due to contact with the protective cover, during movement of the needle shaft or while the shaft is at rest.

### SUMMARY OF THE INVENTION

It is thus the object of the present invention to overcome the disadvantages and shortcomings of prior arterial and similar needle protective safety covers.

It is the object of the present invention to provide a protective cover for an arterial needle which provides an efficient and effective enclosure of the needle tip.

It is another object of the present invention to provide a protective cover for an arterial needle which can be used to securely enclose the tip of the needle and then can be removed from the tip to uncover the needle and allow needle reuse without interference with the cover or contact between the cover and needle tip.

It is a further object of the present invention to provide a protective cover for an arterial needle which is readily and easily retractable along the shaft of the needle.

It is still another object of the present invention to provide a protective cover for an arterial needle which allows for the safe and easy placement of the cover onto and off the needle tip without interference with the cover or contact between the cover and needle tip.

It is a further object of the present invention to provide a protective cover for an arterial needle which, when positioned over the needle tip, ensures for a positive and secure locked enclosure of the tip by directly clamping the needle shaft.

It is still another object of the present invention to provide a protective cover for an arterial needle which permits the needle to be autoclaved.

It is another object of the present invention to provide a protective cover for an arterial needle which is relatively simple, but durable in design, and which can be manufactured economically.

It is a further object of the present invention to provide a protective cover for an arterial needle which is not restricted by the size of the needle, since it can be used on needles of any length and needle gauge.

These and other objects are accomplished by the present invention, a protective cover for arterial needles which is effective in covering the needle tip, but which also can be retracted along the needle shaft to uncover and reopen the needle without interference with the cover or causing contact damage between the cover and the needle tip. The cover has a sheath section with a through channel into which the shaft is slideably received. The cover's sheath section travels substantially along the shaft. This movement is controlled by an actuator control knob connected to a biased locking spring clip or leaf spring located within the sheath section. The locking spring has dual openings through which the shaft travels. When the cover is secured in position on the needle shaft, e.g. when the needle is to be used or when the needle tip is enclosed, the openings are slightly misaligned. This causes locking of the resilient shaft within the protective cover. Pressing the control knob aligns the openings, which unlocks the needle tip and allows the cover to be slid along the shaft.

Novel features which are considered as characteristic of the invention are set forth in particular in the dependent claims. The invention, itself, however, both as to its design, construction and use, together with the additional features and advantages thereof, are best understood upon review of the following detailed description with reference to the accompanying drawings.

### BRIEF DESCRIPTION OF THE DRAWINGS

FIG. 1 is an elevation view of the protective cover of the present invention positioned adjacent to the base of an arterial needle.

FIG. 2 is an elevation view of the protective cover of the present invention shown being slid along the shaft of an arterial needle.

FIG. 3 is an elevation, cross-sectional view of the protective cover of the present invention enclosing and locking the tip of an arterial needle.

FIG. 4 is an elevation, cross-sectional view of the protective cover of the present invention as the tip of an arterial needle is being unlocked.

FIG. 5 is a front view of the locking spring of the protective cover of the present invention in position to secure the shaft of an arterial needle to the cover.

FIG. 6 is a front view of the locking spring of the protective cover of the present invention in position to allow movement of the cover along the shaft of an arterial needle.

### DETAILED DESCRIPTION OF THE INVENTION

Arterial needle 1 comprises base member 2 and needle shaft 4 with needle tip 6. Cover 10 is configured to be slideably mounted on shaft 4. Cover 10 has a sheath section 12 with through channel 14 extending therethrough. Sheath section 12 further comprises outwardly extending actuator control knob 8 connected to biased locking spring, spring clip or leaf spring 16. Locking spring 16 has two legs 17 and 18 and dual openings 19 and 20. Opening 19 extends through leg 17 and opening 20 extends through leg 18. Openings 19 and 20 are slightly misaligned, in the normal biased position of locking spring 16, as seen in FIG. 5. When a compressive force is applied to locking spring 16, openings 19 and 20 become aligned as seen in FIG. 6. The lower end of leg 18 provides the connection between locking spring 16 and control knob 8.

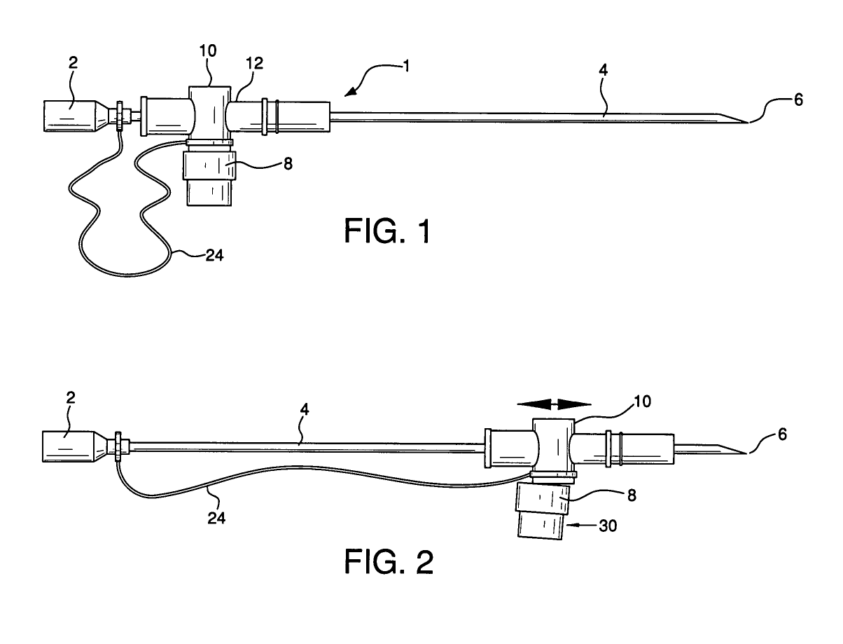

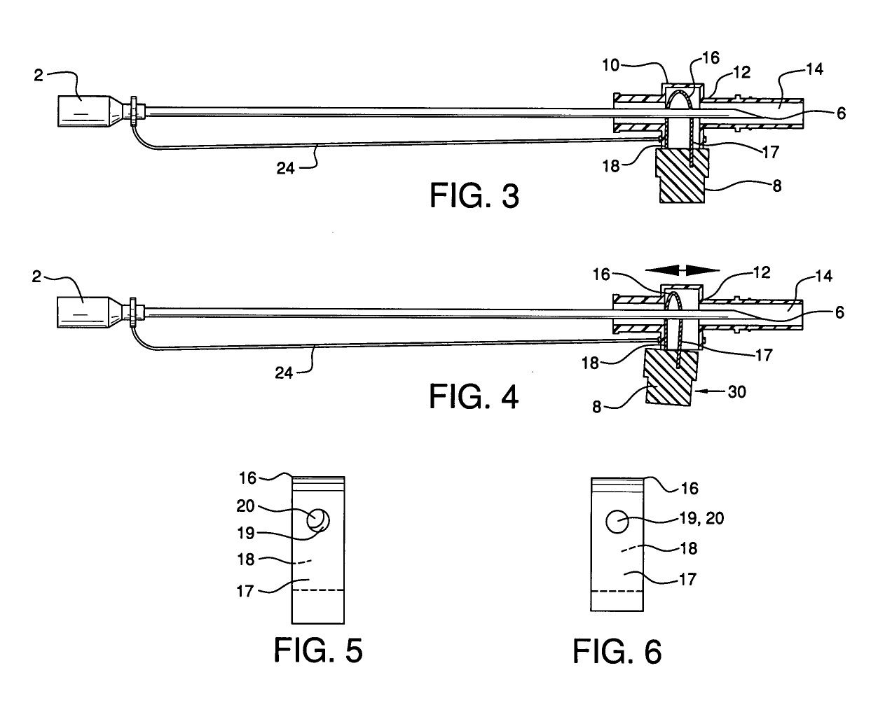

When needle 1 is being used, cover 10 is positioned adjacent to base member 2, as seen in FIG. 1. Needle shaft 4 extends through channel 14 of sheath section 12, as described above. When maintained in this position, locking spring 16 is normally biased, with openings 19 and 20 slightly misaligned. The resilient nature of shaft 4 results in the shaft being flexed slightly, within misaligned openings 19 and 20. This flexed configuration locks shaft 4 within locking spring 16 and immobilizes cover 10 in relation to the shaft.

When cover 10 is to be slid along shaft 4 to enclose or uncover needle tip 6, control knob 8 is depressed, such as is designated by arrow 30 in FIG. 4. This pressure 30 compels leg 17 to move towards leg 18, which serves to align openings 19 and 20, thus allowing shaft 4 to freely move within the openings. As long as pressure 30 is applied to control knob 8, openings 19 and 20 remain aligned and cover 10 is free to slideably move along shaft 4 to enclose tip 6 or to slide towards base 2 when needle 1 is to be used.

When pressure 30 is released, locking spring 16 returns to its naturally biased position, i.e. legs 17 and 18 spread apart, openings 19 and 20 again become slightly misaligned, and cover 10 is again locked in position on shaft 4.

Tether strap 24 connects cover 10 with base member 2. Tether strap 24 is specifically sized to limit the movement of cover 10 along shaft 4 and to ensure that the cover can not travel off the shaft.

It can thus be seen that cover 10 provides permanently attached, user-friendly, yet effective safety enclosures for arterial and similar needles. They further permit the needles to be reopened for immediate reuse or to allow the needles to be autoclaved and used again. Significantly, the covers are configured to easily enclose the tops of needles without risk of possible damage to the tips caused by contact with the cover during movement of the needle shaft within the cover. The covers themselves are shaped for convenient and efficient use, however it is contemplated that a variety of cover shapes and configurations can be employed without departing from the spirit and intent of the invention. The scope of the invention is not to be considered restricted by the shape of the cover.

Similarly, while a single tether 24 is shown, it is envisioned that two or more tethers may be advantageously employed, as necessary, to ensure that the cover is moved the requisite distance along the needle shaft.

It is also contemplated that a gate or wall could be placed in front of needle tip 6 once needle shaft 4 is pulled back into cover 10. This could be done mechanically or automatically. Once such method would be to configure locking spring 16 in a sideway “z”, such that the front of the spring would not have a hole and would create a wall through which the needle could not pass. Another option would be to simply placed a cap over the tip. A third option would be to have needle shaft 4 off-center and install a rotating cap with an end hole, also off-center, whereby when the cap is rotated, it would seal off the open end to allow or prevent passage of the needle.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A retractable protective cover for an arterial needle with a base member and a needle shaft with a tip extending therefrom, said cover comprising:
(a) sheath means slideably mounted on the needle shaft for forming a protective enclosure around the tip of the needle shaft, said sheath means comprising channel means for receiving the needle shaft and for allowing slideable movement of the cover along the needle shaft; and
(b) actuator means mounted in spaced relation the sheath means for controlling the sliding movement of the sheath means between the base member and the tip of the needle shaft and for locking the cover over the tip of the needle shaft, said actuator means comprising opening means which, when aligned with the channel means, allows the cover to slideably move along the needle shaft within the channel means and the opening means and when not aligned with the channel means, secures the cover in position over the tip of the needle shaft.

2. The protective cover as in claim 1 wherein the biasing means comprises dual openings through which the needle shaft passes.

3. The protective cover as in claim 1 wherein the control knob is connected to the biasing means.

4. The protective cover as in claim 2 whereby the application of pressure on control knob aligns the dual openings to allow the cover to slideably move along the needle shaft within the channel means.

5. The protective cover as in claim 2 wherein the needle shaft is locked within the sheath means when the dual openings are not in alignment.

6. The protective cover as in claim 3 whereby upon application of pressure on the control knob in opposition to the expansive force of the biasing means, the cover can be unlocked and retractably slid along the needle shaft.

7. The protective cover as in claim 3 whereby the application of pressure on the control knob aligns the dual openings to allow the cover to slideably move along the needle shaft within the channel means.

8. the protective cover as in claim 3 wherein the needle shaft is locked within the sheath means when the dual openings are not in alignment.

9. The protective cover as in claim 1 further comprising tether means between the cover and the base member for limiting the movement of the cover in relation to the base member.

10. The protective cover as in claim 1 wherein the biasing means comprises a leaf spring.

11. The protective cover as in claim 1 wherein the biasing means comprises a spring clip.

12. A retractable protective cover for an arterial needle with a base member and a needle shaft with a tip extending therefrom, said cover comprising:
(a) A sheath having a channel extending therethrough, said channel configured to receive the needle shaft and for allowing slideable movement of the cover along the needle shaft;
(b) A control knob mounted in spaced relation to the sheath; and
(c) biasing means connected to the control knob for providing an expansive force in relation to the control knob, said biasing means having dual openings through which the needle shaft passes, whereby said biasing means locks the needle shaft within the sheath to secure the cover in position over tip of the needle shaft and, upon application of pressure on the control knob, the biasing means releases the shaft to allow the cover to slideably move along the needle shaft within the channel.

13. The protective cover as in claim 12 further comprising a tether connecting the cover to the base member.

14. The protective cover as in claim 12 whereby upon application of pressure on the control knob in opposition to the expansive force of the biasing means, the cover can be unlocked and retractably slid along the needle shaft.

15. The protective cover as in claim 12 wherein the control knob extends from the sheath.

16. The protective cover as in claim 12 wherein the biasing means comprises a leaf spring.

17. The protective cover as in claim 12 wherein the biasing means comprises a spring clip.
